(19) United States
(12) Patent Application Publication
(10) Pub. No.: US 2010/0035954 A1
(43) Pub. Date: Feb. 11, 2010

(54) ACID ADDITION SALTS OF MUSCARINIC RECEPTOR ANTAGONISTS

(76) Inventors: Mohammad Salman, Princeton, NJ (US); Naresh Kumar, Gurgaon Haryana (IN)

Correspondence Address: Ranbaxy Inc., Intellectual Property Department, 600 College Road East, PRINCETON, NJ, 08540, US

(21) Appl. No.: 11/721,838

(22) PCT Filed: Dec. 15, 2004
(86) PCT No.: PCT/IB04/04142

§ 371 Date: Oct. 16, 2009

(51) Int. Cl. A61K 31/403; C07D 209/52; A61P 11/00; A61P 13/00; A61P 1/00

(52) U.S. Cl. 514/412; 548/515

(57) ABSTRACT

Provided herein are acid addition salts of muscarinic receptor antagonists. Such acid addition salts are muscarinic receptor antagonists, which are useful, among other uses, for the treatment of various diseases of the respiratory, urinary and gastrointestinal systems mediated through muscarinic receptors. Also provided herein are processes for the preparation of acid addition salts, pharmaceutical compositions thereof, and methods of treating diseases mediated through muscarinic receptors.

### FIELD OF THE INVENTION

Provided herein are acid addition salts of muscarinic receptor antagonists. Such acid addition salts are muscarinic receptor antagonists, which are useful, among other uses, for the treatment of various diseases of the respiratory, urinary and gastrointestinal systems mediated through muscarinic receptors. Also provided herein are processes for the preparation of acid addition salts, pharmaceutical compositions thereof, and methods of treating diseases mediated through muscarinic receptors.

### BACKGROUND OF THE INVENTION

Muscarinic antagonists, such as atropine, have been known for over a century, but little progress has been made in the discovery of receptor subtype-selective compounds, making it difficult to assign specific functions to individual receptors. Although classical muscarinic antagonists, such as atropine, are potent bronchodilators, their clinical utility is limited due to high incidence of peripheral and central adverse effects, such as tachycardia, blurred vision, dryness of mouth, constipation, dementia etc. Subsequent development of the quarterly derivatives of atropine, such as ipratropium bromide, are better tolerated than parenterally administered options. However, most of these are not ideal anti-cholinergic bronchodilators because they lack of selectivity for muscarinic receptor sub-types, resulting in dose-limiting side-effects, such as thirst, nausea, mydriasis and those associated with the heart, such as tachycardia mediated by the M₂ receptor.

Anti-muscarinic agents, such as oxybutynin and tolterodine, that act non-selectively on muscarinic receptors have been used for many years to treat bladder hyperactivity. The clinical effectiveness of these agents has been limited because of side effects, such as dry mouth, blurred vision and constipation. Muscarinic receptor antagonists have been disclosed in international (PCT) applications WO 04/018422, 04/014853, 04/014363, 04/004629, 04/005252, 04/052857, 04/056811, 04/056810 and 04/056767 and in the references cited therein and also in co-pending International (PCT) Patent Application Serial No. PCT/IB2004/000008.

Accordingly, there remains a need for new highly selective muscarinic antagonists that can interact with distinct receptor subtypes while avoiding adverse effects.

### SUMMARY OF THE INVENTION

Provided herein are acid addition salts of muscarinic receptor antagonists of Formula I,

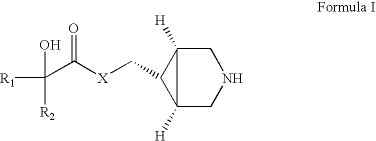

which are useful as therapeutic or prophylactic agents for the treatment of various diseases of the respiratory, urinary and gastrointestinal systems, and process for the synthesis of the compounds.

The invention also includes the enantiomers, diastereomers, N-oxides, prodrugs, polymorphs and pharmaceutically acceptable solvates of these compounds as well as metabolites having the same type of activity.

The pharmaceutical compositions comprising the compounds of the present invention, their prodrugs, metabolites, enantiomers, diastereomers, N-oxides, polymorphs, solvates alone or in combination with a pharmaceutically acceptable carrier, optionally included excipients and diluents that are useful for the treatment of various diseases of the respiratory, urinary and gastrointestinal systems.

Other aspects of the invention will be set forth in the description which follows, and in part will be apparent from the description or may be learnt by the practice of the invention.

In accordance with one aspect, there are provided acid addition salts of muscarinic receptor antagonists of Formula I, having the structure of Formula II:

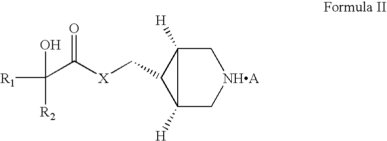

pharmaceutically acceptable solvates, esters, enantiomers, diastereomers, N-oxides, prodrugs, polymorphs and metabolites thereof, wherein, R₁ can be optionally substituted phenyl; R₂ can be optionally substituted alkyl, optionally substituted phenyl or optionally substituted cycloalkyl (wherein the optional sustituent can be halogens); X can be —NH—, —O— or —NMe; A can be organic acid selected from acetic acid, succinic acid, maleic acid, trifluoroacetic acid, oxalic acid, citric acid, malonic acid, adipic acid, ascorbic acid, camphoenic acid, nicotinic acid, butyric acid, tartaric acid, lactic acid and glucuronic acid or inorganic acid selected from hydrochloric acid, hydrobromic acid, phosphoric acid, sulfuric acid, nitric acid, boric acid and perchloric acid with the proviso that A can not be tartaric acid when R₁ and R₂ are phenyl and X is —NMe .

In accordance with one particular embodiment, there are provided hydrochloric acid salts of Formula I having structure of Formula III,

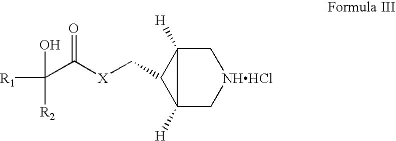

pharmaceutically acceptable solvates, esters, enantiomers, diastereomers, N-oxides, prodrugs, polymorphs and metabolites thereof, wherein R₁, R₂ and X are the same as defined earlier.

In accordance with another embodiment, there are provided maleic acid salts of Formula I having structure of Formula IV,

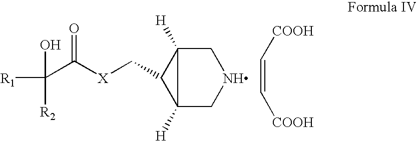

pharmaceutically acceptable solvates, esters, enantiomers, diastereomers, N-oxides, prodrugs, polymorphs and metabolites therof, wherein R₁, R₂ and X are the same as defined earlier.

In accordance with another embodiment, there are provided succinic acid salts of Formula I having structure of Formula V,

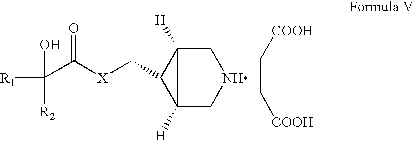

pharmaceutically acceptable solvates, esters, enantiomers, diastereomers, N-oxides, prodrugs, polymorphs and metabolites thereof, wherein R₁, R₂ and X are the same as defined earlier.

In accordance with another embodiment, there are provided acetic acid salts of Formula I having structure of Formula VI,

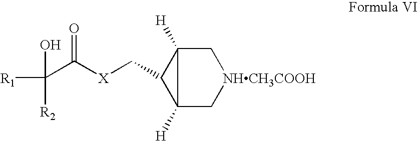

pharmaceutically acceptable solvates, esters, enantiomers, diastereomers, N-oxides, prodrugs, polymorphs and metabolites thereof, wherein R₁, R₂ and X are the same as defined earlier.

In accordance with another embodiment, there are provided tartarate acid salts of Formula I, having structure of Formula VII,

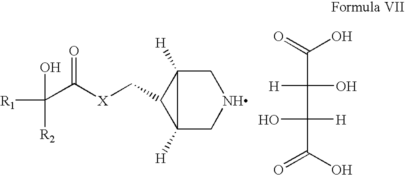

pharmaceutically acceptable solvates, esters, enantiomers, diastereomers, N-oxides, prodrugs, polymorphs and metabolites thereof, wherein R₁, R₂ and X are the same as defined earlier with the proviso that R₁ and R₂ are not phenyl and X is not —NMe.

In accordance with another aspect, there is provided a method for treatment or prophylaxis of an animal or a human suffering from a disease or disorder of the respiratory, urinary and gastrointestinal systems, wherein the disease or disorder is mediated through muscarinic receptors.

In accordance with another aspect, there is provided a method for treatment or prophylaxis of an animal or a human suffering from a disease or disorder associated with muscarinic receptors, comprising administering to a patient in need thereof, an effective amount for muscarinic receptor antagonist compound as described above.

In accordance with yet another aspect, there is provided a method for treatment or prophylaxis of an animal or a human suffering from a disease or disorder of the respiratory system such as bronchial asthma, chronic obstructive pulmonary disorders (COPD), pulmonary fibrosis, etc.; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with another aspect, there is provided a process for preparing the acid addition salts as described above.

As used herein the term “alkyl” refers to straight or branched saturated hydrocarbon having one to six carbon atom (s). Examples of alkyl include, but are not limited to, methyl, ethyl, propyl, isopropyl, butyl and pentyl, and the like.

As used herein the term “cycloalkyl” refers to saturated carbocyclic ring having three to seven carbon atoms. Examples of cycloalkyl include, but are not limited to, cyclopropyl, cyclobutyl, cyclopentyl and cyclohexyl, and the like.

### DETAILED DESCRIPTION OF THE INVENTION

The compounds of the present invention may be prepared by techniques well known in the art and familiar to the average synthetic organic chemist. In addition, the compounds of the present invention may be prepared by the following reaction sequence:

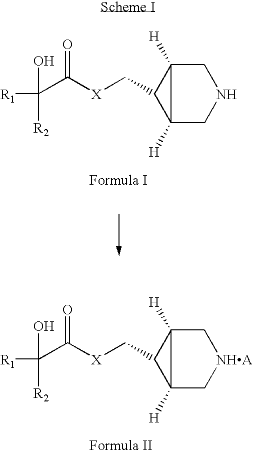

The compounds of Formula II can be prepared by following Scheme I. Accordingly, reacting a compound of Formula I (prepared by following the methods mentioned in WO 04/005252 and in our co-pending International (PCT) Patent Application Serial No. PCT/IB2004/000008) with an organic or inorganic acid to give a compound of Formula II (wherein A, R₁, R₂ and X are the same as defined earlier), which is then isolated by using the methods well known to one ordinary skilled in art.

The reaction of a compound of Formula I with an organic or inorganic acid to give a compound of Formula II can be carried out in a solvent that has no adverse effect on the reaction and it can dissolve the starting material and the acid to some extent. Examples of such solvents include aliphatic hydrocarbons, for example, hexane, cyclohexane, pentane; heptane; aromatic hydrocarbons, for example, benzene, toluene, xylene; halogenated hydrocarbons, for example, dichloromethane, dichloroethane, chloroform, carbon tetrachloride; ethers, for example, diethyl ether, tetrahydrofuran, dioxane; ketones, for example, acetone, diethyl ketone; esters, for example, ethyl acetate, propyl acetate; nitriles, for example, acetonotrile, propionitrile; or alcoholic solvents, for example, methanol, ethanol, isopropanol.

The solvent used to dissolve compound of Formula I can be the same as or different from the solvent employed for acid solution provided that the choice of the solvent to dissolve the acid does not adversely affect the solubility of compound of Formula I when the two solutions are added together during the treatment step.

Acid can be added to the compound in any proportion with respect to compound of Formula I which results in the formation of at least some of the desired acid addition salt.

The solution of compound of Formula I (free base) can be treated with an organic or inorganic acid directly, for example, by bubbling gaseous acid into the solution or acid can first be dissolved in a solvent and then added as a solution of acid. The acid solution can be added all at once or can be added in two or more portions, or can be added incrementally.

The reaction of compound of Formula I to give a compound of Formula II can be conducted at any temperature at which compound of Formula I is soluble in the chosen solvent.

Particular compounds are:
-(1α, 5α, 6α)-3-azabicyclo[3.1.0]hex-6-ylmethyl hydroxy (diphenyl) acetate hydrochloride (Compound No. 1),
-N-[(1α, 5α, 6α)-3-azabicyclo[3.1.0.]hex-6-yl-methyl]-2-phenyl-2-hydroxy-2-(N-methyl) phenyl acetamide succinate (Compound No. 2),
-N-[(1α,5α,6α)-3-azabicyclo[3.1.0.]hex-6-yl-methyl]-2-phenyl-2-hydroxy-2-(N-methyl) phenyl acetamide maleate (Compound No. 3),
-N-[(1α, 5α, 6α)-3-azabicyclo[3.1.0.]hex-6-yl-methyl]-2-phenyl-2-hydroxy-2-(N-methyl) phenyl acetamide acetate (Compound No. 4),
-N-[(1α, 5α, 6α)-3-azabicyclo[3.1.0.]hex-6-yl-methyl]-2-phenyl-2-hydroxy-2-(N-methyl) phenyl acetamide trifluoro acetate (Compound No. 5),
-(2R)-N-[(1α, 5α, 6α)-3-azabicyclo[3.1.0]hex-6-ylmethyl]-2-(3,3-difluorocyclopentyl)-2-hydroxy-2-phenylacetamide tartarate (Compound No. 6),
-(2R)-N-[(1α, 5α, 6α)-3-azabicyclo[3.1.0]hex-6-ylmethyl]-2-(3,3-difluorocyclopentyl)-2-hydroxy-2-phenylacetamide oxalate (Compound No. 7),
-(2R)-N-[(1α, 5α, 6α)-3-azabicyclo[3.1.0]hex-6-ylmethyl]-2-(3,3-difluorocyclopentyl)-2-hydroxy-2-phenylacetamide citrate (Compound No. 8),
-(2R)-N-[(1α, 5α, 6α)-3-azabicyclo[3.1.0]hex-6-ylmethyl]-2-(3,3-difluorocyclopentyl)-2-hydroxy-2-phenylacetamide malonate (Compound No. 9),
-(2R)-N-[(1α, 5α, 6α)-3-azabicyclo[3.1.0]hex-6-ylmethyl]-2-(3,3-difluorocyclopentyl)-2-hydroxy-2-phenylacetamide adipate (Compound No. 10),
-N-[(1α, 5α, 6α)-3-azabicyclo[3.1.0.]hex-6-yl-methyl]-2-phenyl-2-hydroxy-2-(N-methyl) phenyl acetamide hydrochloride (Compound No. 11),
-(1α, 5α, 6α)-3-azabicyclo[3.1.0]hex-6-ylmethyl hydroxy (diphenyl) acetate ascorbate (Compound No. 12),
-(1α, 5α, 6α)-3-azabicyclo[3.1.0]hex-6-ylmethyl hydroxy (diphenyl) acetate camphorate (Compound No. 13),
-(1α, 5α, 6α)-3-azabicyclo[3.1.0]hex-6-ylmethyl hydroxy (diphenyl) acetate nicotionate (Compound No. 14),
-(1α, 5α, 6α)-3-azabicyclo[3.1.0]hex-6-ylmethyl hydroxy (diphenyl) acetate butyrate (Compound No. 15),
-1α, 5α, 6α)-3-azabicyclo[3.1.0]hex-6-ylmethyl hydroxy (diphenyl) acetate lactate (Compound No. 16),
-N-[(1α, 5α, 6α)-3-azabicyclo[3.1.0]hex-6-ylmethyl]-2-hydroxy-2,2-diphenylacetamide hydrochloride (Compound No. 17),
-N-[(1α, 5α, 6α)-3-azabicyclo[3.1.0]hex-6-ylmethyl]-2-hydroxy-2,2-diphenylacetamide glucuronate (Compound No. 18),
-N-[(1α, 5α, 6α)-3-azabicyclo[3.1.0]hex-6-ylmethyl]-2-hydroxy-2,2-diphenylacetamide hydrobromide (Compound No. 19),
-N-[(1α, 5α, 6α)-3-azabicyclo[3.1.0]hex-6-ylmethyl]-2-hydroxy-2,2-diphenylacetamide phosphorate (Compound No. 20),
-N-[(1α, 5α, 6α)-3-azabicyclo[3.1.0]hex-6-ylmethyl]-2-cyclohexyl-2-hydroxy-2-phenylacetamide hydrochloride (Compound No. 21),
-N-[(1α, 5α, 6α)-3-azabicyclo[3.1.0]hex-6-ylmethyl]-2-cyclohexyl-2-hydroxy-2-phenylacetamide maleate (Compound No. 22),
-N-[(1α, 5α, 6α)-3-azabicyclo[3.1.0]hex-6-ylmethyl]-2-cyclohexyl-2-hydroxy-2-phenylacetamide sulfonate (Compound No. 23),
-N-[(1α, 5α, 6α)-3-azabicyclo[3.1.0]hex-6-ylmethyl]-2-(4-fluorophenyl)-2-hydroxy-2-phenylacetamide tartarate (Compound No. 24),
-N-[(1α, 5α, 6α)-3-azabicyclo[3.1.0]hex-6-ylmethyl]-2-(4-fluorophenyl)-2-hydroxy-2-phenylacetamide succinate (Compound No. 25),
-N-[(1α, 5α, 6α)-3-azabicyclo[3.1.0]hex-6-ylmethyl]-2-(4-fluorophenyl)-2-hydroxy-2-phenylacetamide maleate (Compound No. 26),
-(1α, 5α, 6α)-3-azabicyclo[3.1.0]hex-6-ylmethyl 3-ethyl-2-hydroxy-2-phenylpentanoate hydrochloride (Compound No. 27),
-(1α, 5α, 6α)-3-azabicyclo[3.1.0]hex-6-ylmethyl 3-ethyl-2-hydroxy-2-phenylpentanoate maleate (Compound No. 28),
-(1α, 5α, 6α)-3-azabicyclo[3.1.0]hex-6-ylmethyl 3-ethyl-2-hydroxy-2-phenylpentanoate nitrate (Compound No. 29),
-(1α, 5α, 6α)-3-azabicyclo[3.1.0]hex-6-ylmethyl 3-ethyl-2-hydroxy-2-phenylpentanoate borate (Compound No. 30),
-N-[(1α, 5α, 6α)-3-azabicyclo[3.1.0]hex-6-ylmethyl]-2-(4-fluorophenyl)-2-hydroxy-2-phenylacetamide perchlorate (Compound No. 31),
-(1α, 5α, 6α)-3-azabicyclo[3.1.0]hex-6-ylmethyl 2-hydroxy-3-methyl-2-phenylbutanoate hydrochloride (Compound No. 32),
-(1α, 5α, 6α)-3-azabicyclo[3.1.0]hex-6-ylmethyl 2-hydroxy-3-methyl-2-phenylbutanoate succinate (Compound No. 33),
-(1α, 5α, 6α)-3-azabicyclo[3.1.0]hex-6-ylmethyl 2-hydroxy-3-methyl-2-phenylbutanoate hydrobromide (Compound No. 34),

and their pharmaceutically acceptable solvates, esters, enantiomers, diastereomers, N-oxides, prodrugs, polymorphs and metabolites.

TABLE I

| | |
|---|---|
| | Formula II |
| 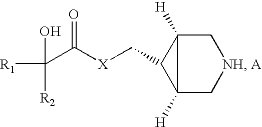 | |

| Com- | | | | |
|---|---|---|---|---|
| pound | | | | |
| No. | R₁ | R₂ | X | A |
| 1. | phenyl | phenyl | —O— | hydrochloric |
| | | | | acid |
| 2. | phenyl | phenyl | —NMe | succinic acid |
| 3. | phenyl | phenyl | —NMe | maleic acid |
| 4. | phenyl | phenyl | —NMe | acetic acid |
| 5. | phenyl | phenyl | —NMe | trifluoro |
| | | | | acetic acid |
| 6. | phenyl | 3,3-difluorocyclopentyl | —NH— | tartaric acid |
| 7. | phenyl | 3,3-difluorocyclopentyl | —NH— | oxalic acid |
| 8. | phenyl | 3,3-difluorocyclopentyl | —NH— | citric acid |
| 9. | phenyl | 3,3-difluorocyclopentyl | —NH— | malonic acid |
| 10. | phenyl | 3,3-difluorocyclopentyl | —NH— | adipic acid |
| 11. | phenyl | phenyl | —NMe | hydrochloric |
| | | | | acid |
| 12. | phenyl | phenyl | —O— | ascorbic acid |
| 13. | phenyl | phenyl | —O— | camphoric acid |
| 14. | phenyl | phenyl | —O— | nicotinic acid |
| 15. | phenyl | phenyl | —O— | butyric acid |
| 16. | phenyl | phenyl | —O— | lactic acid |
| 17. | phenyl | phenyl | —NH— | hydrochloric |
| | | | | acid |
| 18. | phenyl | phenyl | —NH— | glucuronic acid |
| 19. | phenyl | phenyl | —NH— | hydrobromic |
| | | | | acid |
| 20. | phenyl | phenyl | —NH— | phosphoric acid |
| 21. | phenyl | cyclohexyl | —NH— | hydrochloric |
| | | | | acid |
| 22. | phenyl | cyclohexyl | —NH— | maleic acid |
| 23. | phenyl | cyclohexyl | —NH— | sulfonic acid |
| 24. | phenyl | 4-fluorophenyl | —NH— | tartaric acid |
| 25. | phenyl | 4-fluorophenyl | —NH— | succinic acid |
| 26. | phenyl | 4-fluorophenyl | —NH— | maleic acid |
| 27. | phenyl | 3-pentyl | —O— | hydrochloric |
| | | | | acid |
| 28. | phenyl | 3-pentyl | —O— | maleic acid |
| 29. | phenyl | 3-pentyl | —O— | nitric acid |
| 30. | phenyl | 3-pentyl | —O— | boric acid |
| 31. | phenyl | 4-fluorophenyl | —NH— | perchloric acid |
| 32. | phenyl | isopropyl | —O— | hydrochloric |
| | | | | acid |
| 33. | phenyl | isopropyl | —O— | succinic acid |
| 34. | phenyl | isopropyl | —O— | hydrobromic |
| | | | | acid |

Because of their valuable pharmacological properties, the compounds of the present invention may be administered to an animal for treatment orally, or by parenteral route. The pharmaceutical compositions of the present invention are preferably produced and administered in dosage units, each unit containing a certain amount of at least one compound of the invention and/or at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The present invention also includes within its scope prodrugs of these agents. In general, such prodrugs will be functional derivatives of these compounds, which are readily convertible in vivo into the required compound. Conventional procedures for the selection and preparation of suitable prodrug derivatives are described, for example, in “Design of Prodrugs”, ed. H Bundgaard and, Elsevier, 1985.

The present invention also includes metabolites, which become active upon introduction into the biological system.

The compounds of the invention possess two chiral centers, they may, therefore, exist as enantiomers and diastereomers. It is to be understood that all such isomers and racemic mixtures therefore are encompassed within the scope of the present invention.

The crystalline or amorphous forms of compounds disclosed herein may exist as polymorphs and as such are intended to be included in the present invention.

While the present invention has been described in terms of its specific embodiments, certain modifications and equivalents will be apparent to those skilled in the art and are included within the scope of the present invention.

The examples mentioned below demonstrate the general synthetic procedure as well as the specific preparation of the preferred compound. The examples are provided to illustrate the details of the invention and should not be constrained to limit the scope of the present invention.

### EXAMPLES

### Example 1

### Preparation of Compound of Formula II (when A is organic acid)

### General Procedure:

To a solution of compound of Formula I in alcoholic solvent corresponding acid (1 equiv.) was added and the solution is stirred for about 1 h. The solvent is removed to ⅕th of the total volume. The salt is precipitated by addition of a non-polar solvent. The salt is filtered off, washed with ether and dried under vacuum.

The following compounds can be prepared following the above general procedure
-N-[(1α, 5α, 6α)-3-azabicyclo[3.1.0.]hex-6-yl-methyl]-2-phenyl-2-hydroxy-2-(N-methyl) phenyl acetamide succinate (Compound No. 2),
-N-[(1α, 5α, 6α)-3-azabicyclo[3.1.0.]hex-6-yl-methyl]-2-phenyl-2-hydroxy-2-(N-methyl) phenyl acetamide maleate (Compound No. 3),
-N-[(1α, 5α, 6α)-3-azabicyclo[3.1.0.]hex-6-yl-methyl]-2-phenyl-2-hydroxy-2-(N-methyl) phenyl acetamide acetate (Compound No. 4),
-N-[(1α, 5α, 6α)-3-azabicyclo[3.1.0.]hex-6-yl-methyl]-2-phenyl-2-hydroxy-2-(N-methyl) phenyl acetamide trifluoro acetate (Compound No. 5),
-(2R)-N-[(1α, 5α, 6α)-3-azabicyclo[3.1.0]hex-6-ylmethyl]-2-(3,3-difluorocyclopentyl)-2-hydroxy-2-phenylacetamide oxalate (Compound No. 7),
-(2R)-N-[(1α, 5α, 6α)-3-azabicyclo[3.1.0]hex-6-ylmethyl]-2-(3,3-difluorocyclopentyl)-2-hydroxy-2-phenylacetamide citrate (Compound No. 8),
-(2R)-N-[(1α, 5α, 6α)-3-azabicyclo[3.1.0]hex-6-ylmethyl]-2-(3,3-difluorocyclopentyl)-2-hydroxy-2-phenylacetamide malonate (Compound No. 9),
-(2R)-N-[(1α, 5α, 6α)-3-azabicyclo[3.1.0]hex-6-ylmethyl]-2-(3,3-difluorocyclopentyl)-2-hydroxy-2-phenylacetamide adipate (Compound No. 10),
-(1α, 5α, 6α)-3-azabicyclo[3.1.0]hex-6-ylmethyl hydroxy (diphenyl) acetate ascorbate (Compound No. 12),
-(1α, 5α, 6α)-3-azabicyclo[3.1.0]hex-6-ylmethyl hydroxy (diphenyl) acetate camphorate (Compound No. 13),
-(1α, 5α, 6α)-3-azabicyclo[3.1.0]hex-6-ylmethyl hydroxy (diphenyl) acetate nicotionate (Compound No. 14),
-(1α, 5α, 6α)-3-azabicyclo[3.1.0]hex-6-ylmethyl hydroxy (diphenyl) acetate butyrate (Compound No. 15),
-(1α, 5α, 6α)-3-azabicyclo[3.1.0]hex-6-ylmethyl hydroxy (diphenyl) acetate lactate (Compound No. 16),
-N-[(1α, 5α, 6α)-3-azabicyclo[3.1.0]hex-6-ylmethyl]-2-hydroxy-2,2-diphenylacetamide glucuronate (Compound No. 18),
-N-[(1α, 5α, 6α)-3-azabicyclo[3.1.0]hex-6-ylmethyl]-2-cyclohexyl-2-hydroxy-2-phenylacetamide maleate (Compound No. 22),
-N-[(1α, 5α, 6α)-3-azabicyclo[3.1.0]hex-6-ylmethyl]-2-(4-fluorophenyl)-2-hydroxy-2-phenylacetamide succinate (Compound No. 25),
-N-[(1α, 5α, 6α)-3-azabicyclo[3.1.0]hex-6-ylmethyl]-2-(4-fluorophenyl)-2-hydroxy-2-phenylacetamide maleate (Compound No. 26),
-(1α, 5α, 6α)-3-azabicyclo[3.1.0]hex-6-ylmethyl 3-ethyl-2-hydroxy-2-phenylpentanoate maleate (Compound No. 28),
-(1α, 5α, 6α)-3-azabicyclo[3.1.0]hex-6-ylmethyl 2-hydroxy-3-methyl-2-phenylbutanoate succinate (Compound No. 33).

### Example 2

### Preparation of (2R)-N-[(1α, 5α, 6α)-3-azabicyclo[3.1.0]hex-6-ylmethyl]2-(3,3-difluorocyclopentyl)-2-hydroxy-2-phenylacetamide Tartarate (Compound No. 6)

To a solution of (2R)-N-[(1α, 5α, 6α)-3-azabicyclo[3.1.0]hex-6-ylmethyl]-2-(3,3-difluorocyclopentyl)-2-hydroxy-2-phenylacetamide (150 mg) in ethanol, solid L (+) tartaric acid (64 mg ) was added at room temperature and the reaction mixture was stirred for about 4 hours at room temperature. The solvent was evaporated. Dry ether was added to it. White precipitate was observed. The reaction mixture was stirred for about 20 minutes. Supernatant was decanted off and the precipitate was washed with ether to get the product (220 mg).

¹H NMR spectral data showed (DMSO-d₆): δ 7.69 (m, 2H), 7.30 (m, 3H), 4.23 (m, 3H), 4.07 (s, 2H), 3.35 (m, 1H), 3.16 (m, 2H), 2.0-1.8 (m, 8H), 1.29 (m, 1H).

### Example 3

### Preparation of N-[(1α, 5α, 6α)-3-azabicyclo[3.1.0]hex-6-ylmethyl]-2-(4-fluorophenyl-2-hydroxy-2-phenylacetamide Tartarate (Compound No. 24)

To a solution of N-[(1α, 5α, 6α)-3-azabicyclo[3.1.0]hex-6-ylmethyl]-2-(4-fluorophenyl)-2-hydroxy-2-phenylacetamide (225 mg) in ethanol, solid L (+) tartaric acid (99 mg) was added at room temperature and the reaction mixture was stirred for about 4 hours at room temperature. The solvent was evaporated. Dry ether was added to it. White precipitate was observed. The reaction mixture was stirred for about 20 minutes. Supernatant was decanted off and the precipitate was washed with ether to get the product (313 mg). The compound exhibited a melting point of 108.3-111.5° C.

¹H NMR spectral data showed (CD₃OD): δ 7.44-7.27 (m, 7H), 7.01 (m, 2H), 4.42 (s, 2H), 3.28 (m, 4H), 3.18 (m, 2H), 1.74 (m, 2H), 1.09 (m, 1H).

### Example 4

### Preparation of (1α, 5α, 6α)-3-azabicyclo[3.1.0]hex-6-ylmethyl hydroxy (diphenyl) Acetate Hydrochloride (Compound No. 1)

(1α, 5α, 6α)-3-azabicyclo[3.1.0]hex-6-ylmethyl hydroxy (diphenyl) acetate (1.3 g) was dissolved in dichloromethane (10 ml). Ethanolic hydrochloric acid (4N, 1.18 ml) was added to it. The solvent was evaporated to get gummy solid. Hexane was added to the solid. The solvent was evaporated to get free-flowing product (1.36 g).

The following compounds are prepared by following the same procedure
-N-[(1α, 5α, 6α)-3-azabicyclo[3.1.0.]hex-6-yl-methyl]-2-phenyl-2-hydroxy-2-(N-methyl) phenyl acetamide hydrochloride (Compound No. 11) (m.p.: 203-205.7° C.)
-N-[(1α, 5α, 6α)-3-azabicyclo[3.1.0]hex-6-ylmethyl]-2-hydroxy-2,2-diphenylacetamide hydrobromide (Compound No. 19),
-N-[(1α, 5α, 6α)-3-azabicyclo[3.1.0]hex-6-ylmethyl]-2-hydroxy-2,2-diphenylacetamide phosphorate (Compound No. 20),
-N-[(1α, 5α, 6α)-3-azabicyclo[3.1.0]hex-6-ylmethyl]-2-cyclohexyl-2-hydroxy-2-phenylacetamide sulfonate (Compound No. 23),
-(1α, 5α, 6α)-3-azabicyclo[3.1.0]hex-6-ylmethyl 3-ethyl-2-hydroxy-2-phenylpentanoate nitrate (Compound No. 29),
-1α, 5α, 6α)-3-azabicyclo[3.1.0]hex-6-ylmethyl 3-ethyl-2-hydroxy-2-phenylpentanoate borate (Compound No. 30),
-N-[(1α, 5α, 6α)-3-azabicyclo[3.1.0]hex-6-ylmethyl]-2-(4-fluorophenyl)-2-hydroxy-2-phenylacetamide perchlorate (Compound No. 31),
-(1α, 5α, 6α)-3-azabicyclo[3.1.0]hex-6-ylmethyl 2-hydroxy-3-methyl-2-phenylbutanoate hydrobromide (Compound No. 34).

### Example 5

### Preparation of N-[(1α, 5α, 6α)-3-azabicyclo[3.1.0]hex-6-ylmethyl]-2-hydroxy-2-diphenylacetamide hydrochloride (Compound No. 17)

N-[(1α, 5α, 6α)-3-azabicyclo[3.1.0]hex-6-ylmethyl]-2-hydroxy-2,2-diphenylacetamide (0.2 g) was dissolved in dichloromethane (3 ml). Ethanolic hydrochloric acid (3N, 0.23 ml) was added. The reaction mixture was stirred. The solvent was evaporated. Ether was added to the residue and the mixture was stirred again. Ether was evaporated to get the solid product (0.27 g). The compound exhibited a melting point of 108.4-109.5° C.

¹H NMR spectral data showed (CDCl₃): δ 7.41 (m, 10H), 6.90 (bs, 1H), 3.84 (m, 1H), 3.45-3.2 (m, 5H), 1.69 (m, 2H), 1.27 (m, 1H).

### Example 6

### Preparation of N-[(1α, 5α, 6α)-3-azabicyclo[3.1.0]hex-6-ylmethyl]-2-cyclohexyl-2-hydroxy-2-phenylacetamide hydrochloride (Compound No. 21)

N-[(1α, 5α, 6α)-3-azabicyclo[3.1.0]hex-6-ylmethyl]-2-cyclohexyl-2-hydroxy-2-phenylacetamide (1.2 g) was dissolved in dichloromethane. Ethanolic hydrochloric acid (3N, 1.3 ml) was added. The reaction mixture was stirred. The solvent was evaporated. Ether was added to the residue and the mixture was stirred again. Ether was evaporated to get the solid product (1.45 g). The compound exhibited a melting point of 121.6-128.1° C.

¹H NMR spectral data showed (CDCl₃): δ 7.6 (m, 2H), 7.34 (m, 3H), 6.98 (m, 1H), 3.35-3.15 (m, 6H), 2.42 (m, 1H), 1.82-1.61 (m, 3H), 1.22-1.11 (m, 10H). The mass spectrum showed peaks at m/e of: 329 (M+1) (free base)

### Example 7

### Preparation of (1α, 5α, 6α)-3-azabicyclo[3.1.0]hex-6-ylmethyl 3-ethyl-2-hydroxy-2-phenylpentanoate hydrochloride (Compound No. 27)

(1α, 5α, 6α)-3-azabicyclo[3.1.0]hex-6-ylmethyl 3-ethyl-2-hydroxy-2-phenylpentanoate (0.08 g) was dissolved in dichloromethane. Ethanolic hydrochloric acid (3N, 0.086 ml) was added. The reaction mixture was stirred. The solvent was evaporated. Ether was added to the residue and the mixture was stirred again. Ether was evaporated to get the solid product (0.08 g).

¹ H NMR spectral data showed (D₂O): δ 7.70 (m, 2H), 7.48 (m, 3H), 4.13 (m, 2H), 3.45-3.29 (m, 5H), 2.30 (m, 1H), 1.85 (m, 2H), 1.52 (m, 2H), 1.20 (m, 3H), 1.07 (t, J=7.5Hz, 3H), 0.83 (t, J=7.5Hz, 3H).

### Example 8

### Preparation of (1α, 5α, 6α)-3-azabicyclo[3.1.0]hex-6-ylmethyl 2-hydroxy-3-methyl-2-phenylbutanoate hydrochloride (Compound No. 32)

(1α, 5α, 6α)-3-azabicyclo[3.1.0]hex-6-ylmethyl 2-hydroxy-3-methyl-2-phenylbutanoate (0.1 g) was dissolved in dichloromethane. Ethanolic hydrochloric acid (3N, 0.1 ml) was added. The reaction mixture was stirred. The solvent was evaporated. Ether was added to the residue and the mixture was stirred again. Ether was evaporated to get the solid product (0.06 g).

¹H NMR spectral data showed: δ 7.70 (m, 2H), 7.50 (m, 3H), 4.12 (m, 2H), 3.44-3.27 (m, 4H), 2.87 (m, 1H), 1.83 (bs, 2H), 1.22 (m, 1H), 1.06 (d, J=6.6 Hz, 3H), 0.77 (d, J=6.6 Hz, 3H).

### Biological Activity

### Radioligand Binding Assays:

The affinity of test compounds for M₂ and M₃ muscarinic receptor subtypes was determined by [³H]-N-methylscopolamine binding studies, using rat heart and submandibular gland, respectively, as described by Moriya et al., (Life Sci., 1999; 64(25):2351-2358) with minor modifications as follows. The membrane preparation was done with the following modifications: a low spin step of 500 g for 10 minutes at 4° C. was used; the buffer was 20 mM HEPES, 10 mM EDTA, at pH 7.4; the high speed spin was done at 40,000 g and the homogenate was passed through a filter gauge before any spinning. The assay conditions were modified as follows: the assay volume was 250 μL; the incubation time was 3 hours; the PE concentration was 0.1%; the filtermat used was GF/B from Wallac; the scintillant used was Supermix from Wallac; the amount of scintillant was 500 μL/well; and the counter used was a 1450 microbeta PLUS, from Wallac.

Membrane preparation: Submandibular glands and heart were isolated and placed in ice cold homogenising buffer (HEPES 20 mM, 10 mM EDTA, pH 7.4) immediately after sacrifice. The tissues were homogenised in 10 volumes of homogenising buffer and the homogenate was filtered through two layers of wet gauze and filtrate was centrifuged at 500 g for 10 min. The supernatant was subsequently centrifuged at 40,000 g for 20 min. The pellet thus obtained was resuspended in same volume of assay buffer (HEPES 20 mM, EDTA 5 mM, pH 7.4) and were stored at −70° C. until the time of assay.

Ligand binding assay: The compounds were dissolved and diluted in dimethylsulphoxide. The membrane homogenates (150-250 μg protein) were incubated in 250 μl of assay buffer (HEPES 20 mM, pH 7.4) at 24-25° C. for 3 h. Non-specific binding was determined in the presence of 1 μM atropine. The incubation was terminated by vacuum filtration over GF/B fiber filters (Wallac). The filters were then washed with ice-cold 50 mM Tris HCl buffer (pH 7.4). The filter mats were dried and bound radioactivity retained on filters was counted. The IC₅₀ and K₄ were estimated by using the non-linear curve-fitting program using G Pad Prism software. The value of inhibition constant Kᵢ was calculated from competitive binding studies by using Cheng & Prusoff equation (Biochem Pharmacol, 1973; 22:3099-3108), Kᵢ=IC₅₀/(1+L/K₄), where L is the concentration of [³H] NMS used in the particular experiment. pKᵢ=−[log Kᵢ]. The Kᵢ values for compounds disclosed herein, for rat M₃ receptors, were in the range of from about 0.01 to about 2 nM, for example from about 0.01 to about 0.75 nM, or from about 0.01 to about 0.3 nN. The Kᵢ values for compounds disclosed herein, for rat M₂ receptors were in the range of from about 0.01 to about 25 nM, for example from about 0.01 nM to about 8 nM, and from about 0.01 nM to about 1.2 nM.

### Functional Experiments Using Isolated Rat Bladder:

### Methodology:

Animals were euthanized by overdose of urethane and whole bladder was isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; glucose 5.55 and continuously gassed with 95% O₂ and 5% CO₂.

The bladder was cut into longitudinal strips (3 mm wide and 5-6 mm long) and mounted in 10 ml organ baths at 30° C., with one end connected to the base of the tissue holder and the other end connected to a polygraph through a force displacement transducer. Each tissue was maintained at a constant basal tension of 2 g and allowed to equilibrate for 1 hour during which the PSS was changed every 15 min. At the end of equilibration period the stabilization of the tissue contractile response was assessed with 1 μmol/L of Carbachol consecutively for 2-3 times. Subsequently a cumulative concentration response curve to carbachol (10⁻⁹ mol/L to 3×10⁻⁵ mol/L) was obtained. After several washes, once the baseline was achieved, cumulative concentration response curve was obtained in presence of NCE (NCE added 20 min. prior to the second CRC).

The contractile results were expressed as % of control E max. ED50 values were calculated by fitting a non-linear regression curve (Graph Pad Prism). pK_{B} values were calculated by the formula pK_{B}=−log [(molar concentration of antagonist/(dose ratio−1))] where, dose ratio=ED₅₀ in the presence of antagonist/ED₅₀ in the absence of antagonist. The pK_{B} values for rat bladder for particular compounds provided herein were in the range of from about 8.5 to about 9.6, for example, from about 8.7 to about 9.6 or from about 9.1 to about 9.6.

In vivo Experiments Using Anesthetized Rabbit:

The effect of test substances was studied on carbachol-evoked changes on bladder pressure, heart rate and salivation. Male rabbits weighing 1.2-3 kg were anaesthetized with urethane (1.5 g/kg), and administered as a slow intravenous infusion through the marginal ear vein. The tracheae were cannulated to maintain airway patency. Blood pressure was recorded from the femoral artery by means of a Statham P10 EZ pressure transducer connected to a Grass model 7D polygraph. The heart rate was monitored by a tachograph triggered by the pulse wave of blood pressure. The other femoral artery was cannulated for the administration of carbachol. Test compounds and saline were infused intravenously via the femoral vein.

The bladder was exposed through a midline laparotomy and both the ureters were identified, carefully separated and ligated. The ureters were incised proximally to allow free flow of urine from the kidney to the exterior. Bladder neck was gently held and the urethra was traced and separated from the adjoining tissues. PE canula was introduced into the bladder and ligated. The bladder was drained and subsequently filled with 15 ml of warm saline (37° C.). The other end of the intravesical catheter was connected to the Grass model 7D polygraph through a Statham P10 EZ pressure transducer to monitor the bladder pressure. Care was taken to keep the exposed area moist and warm. A period of 30-60 min was allowed for stabilization of parameters subsequent to surgery. Salivation response was assessed by placing preweighed absorbent cotton gauze in the buccal cavity for 2 minutes after carbachol administration. The effect of the compound on carbachol (1.5 μg/kg, intrarterial) induced changes on blood pressure; heart rate and bladder pressure were observed. At least two stable responses were obtained. These responses were considered as 100%. Subsequently, effect of increasing dose of test compound or vehicle (i.v, 12 to 15 min before carbachol challenge) was studied.

The change in bladder pressure, salivation and agonist-induced bradycardia were expressed as % change from pretreatment control. ID₅₀ values (dose required to inhibit 50% of response) were calculated from non-linear curve fitting for sigmoidal dose response curve using Graph Pad Prism software and values were expressed as μg/kg. The in vivo bladder vs. salivary selectivity was in the range of from about 0.7 to about 6 (fold of oxybutynin), for example from about 1.3 to about 6.0.

While the present invention has been described in terms of its specific embodiments, certain modifications and equivalents will be apparent to those skilled in the art and are within the scope of the present invention.

1. Acid addition salts of muscarinic receptor antagonists of Formula I, having the structure of Formula II: 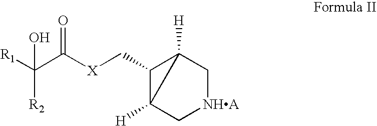
pharmaceutically acceptable solvates, esters, enantiomers, diastereomers, N-oxides, prodrugs, polymorphs and metabolites thereof, wherein,
R₁ is optionally substituted phenyl; R₂ is optionally substituted alkyl, optionally substituted phenyl or optionally substituted cycloalkyl (wherein the optional substituent is halogens); X is —NH—, —O— or —NMe; A is organic acid selected from acetic acid, succinic acid, maleic acid, trifluoroacetic acid, oxalic acid, citric acid, malonic acid, adipic acid, ascorbic acid, camphorenic acid, nicotinic acid, butyric acid, tartaric acid, lactic acid and glucuronic acid or inorganic acid selected from hydrochloric acid, hydrobromic acid, phosphoric acid, sulfuric acid, nitric acid, boric acid and perchloric acid with the proviso that A can not be tartaric acid when R₁ and R₂ are phenyl and X is —NMe.

2. The organic acid according to claim 1 is acetic acid.

3. The organic acid according to claim 1 s succinic acid,

4. The organic acid according to claim 1 is maleic acid.

5. The organic acid according to claim 1 is tartaric acid.

6. The inorganic acid according to claim 1 is hydrochloric acid.

7. A compound, which is:
-(1α, 5α, 6α)-3-azabicyclo[3.1.0]hex-6-ylmethyl hydroxy (diphenyl) acetate hydrochloride (Compound No. 1),
-N-[(1α, 5α, 6α)-3-azabicyclo[3.1.0.]hex-6-yl-methyl]-2-phenyl-2-hydroxy-2-(N-methyl) phenyl acetamide succinate (Compound No. 2),
-N-[(1α, 5α, 6α)-3-azabicyclo[3.1.0.]hex-6-yl-methyl]-2-phenyl-2-hydroxy-2-(N-methyl) phenyl acetamide maleate (Compound No. 3),
-N-[(1α, 5α, 6α)-3-azabicyclo[3.1.0.]hex-6-yl-methyl]-2-phenyl-2-hydroxy-2-(N-methyl) phenyl acetamide acetate (Compound No. 4),
-N-[(1α, 5α, 6α)-3-azabicyclo[3.1.0.]hex-6-yl-methyl]-2-phenyl-2-hydroxy-2-(N-methyl) phenyl acetamide trifluoro acetate(Compound No. 5),
-(2R)-N-[(1α, 5α, 6α)-3-azabicyclo[3.1.0]hex-6-ylmethyl]-2-(3,3-difluorocyclopentyl)-2-hydroxy-2-phenylacetamide tartrate (Compound No. 6),
-(2R)-N-[(1α, 5α, 6α)-3-azabicyclo[3.1.0]hex-6-ylmethyl]-2-(3,3-difluorocyclopentyl)-2-hydroxy-2-phenylacetamide oxalate (Compound No. 7),
-(2R)-N-[(1α, 5α, 6α)-3 -azabicyclo[3.1.0]hex-6-ylmethyl]-2-(3,3-difluorocyclopentyl)-2-hydroxy-2-phenylacetamide citrate (Compound No. 8),
-(2R)-N-[(1α, 5α, 6α)-3-azabicyclo[3.1.0]hex-6-ylmethyl]-2-(3,3 -difluorocyclopentyl)-2-hydroxy-2-phenylacetamide malonate (Compound No. 9),
-(2R)-N-[(1α, 5α, 6α)-3-azabicyclo[3.1.0]hex-6-ylmethyl]-2-(3,3-difluorocyclopentyl)-2-hydroxy-2-phenylacetamide adipate (Compound No. 10),
-N-[(1α, 5α, 6α)-3-azabicyclo[3.1.0.]hex-6-methyl]-2-phenyl-2-hydroxy-2-(N-methyl) phenyl acetamide hydrochloride (Compound No. 11),
-(1α, 5α, 6α)-3-azabicyclo[3.1.0]hex-6-ylmethyl hydroxy (diphenyl) acetate ascorbate (Compound No. 12),
-(1α, 5α, 6α)-3-azabicyclo[3.1.0]hex-6-ylmethyl hydroxy (diphenyl) acetate camphorate (Compound No, 13),
-(1α, 5α, 6α)-3-azabicyclo[3.1.0]hex-6-ylmethyl hydroxy (diphenyl) acetate nicotinate (Compound No. 14),
-(1α, 5α, 6α)-3-azabicyclo[3.1.0]hex-6-ylmethyl hydroxy (diphenyl) acetate butyrate (Compound No. 15),
-(1α, 5α, 6α)-3-azabicyclo[3.1.0]hex-6-ylmethyl hydroxy (diphenyl) acetate lactate (Compound No. 16),
-N-[(1α, 5α, 6α)-3-azabicyclo[3.1.0]hex-6-ylmethyl]-2-hydroxy-2,2-diphenylacetamide hydrochloride (Compound No. 17),
-N-[(1α, 5α, 6α)-3-azabicyclo[3.1.0]hex-6-ylmethyl]-2-hydroxy-2,2-diphenylacetamide glucuronate (Compound No. 18),
-N-[(1α, 5α, 6α)-3-azabicyclo[3.1.0]hex-6-ylmethyl]-2-hydroxy-2,2-diphenylacetamide hydrobromide (Compound No, 19),
-N-[(1α, 5α, 6α)-3-azabicyclo[3.1.0]hex-6-ylmethyl]-2-hydroxy-2,2-diphenylacetamide phosphorate (Compound No. 20),
-N-[(1α, 5α, 6α)-3-azabicyclo[3.1.0]hex-6-ylmethyl]-2-cyclohexyl-2-hydroxy-2-phenylacetamide hydrochloride (Compound No. 21),
-N-[(1α, 5α, 6α)-3-azabicyclo[3.1.0]hex-6-ylmethyl]-2-cyclohexyl-2-hydroxy-2-phenylacetamide maleate(Compound. No. 22),
-N-[(1α, 5α, 6α)-3-azabicyclo[3.1.0]hex-6-ylmethyl]-2-cyclohexyl-2-hydroxy-2-phenylacetamide sulfonate (Compound No. 23),
-N-[(1α, 5α, 6α)-3-azabicyclo[3.1.0]hex-6-ylmethyl]-2-(4-fluorophenyl)-2-hydroxy-2-phenylacetamide tartrate (Compound No. 24),
-N-[(1α, 5α, 6α)-3-azabicyclo[3.1.0]hex-6-ylmethyl]-2-(4-fluorophenyl)-2-hydroxy-2-phenylacetamide succinate (Compound No. 25),
-N-[(1α, 5α, 6α)-3-azabicyclo[3.1.0]hex-6-ylmethyl]-2-(4-fluorophenyl)-2-hydroxy-2-phenylacetamide maleate(Compound No. 26),
-(1α, 5α, 6α)-3-azabicyclo[3.1.0]hex-6-ylmethyl 3-ethyl-2-hydroxy-2-phenylpentanoate hydrochloride (Compound No. 27),
-(1α, 5α, 6α)-3-azabicyclo[3.1.0]hex-6-ylmethyl 3-ethyl-2-hydroxy-2-phenylpentanoate maleate (Compound No. 28),
-(1α, 5α, 6α)-3-azabicyclo[3.1,0]hex-6-ylmethyl 3-ethyl-2-hydroxy-2-phenylpentanoate nitrate (Compound No. 29),
-(1α, 5α, 6α)-3-azabicyclo[3.1.0]hex-6-ylmethyl 3-ethyl-2-hydroxy-2-phenylpentanoate borate (Compound No. 30),
-N-[(1α, 5α, 6α)-3-azabicyclo[3.1.0]hex-6-ylmethyl]-2-(4-fluorophenyl)-2-hydroxy-2-phenylacetamide perchlorate (Compound No. 31),
-(1α, 5α, 6α)-3-azabicyclo[3.1.0]hex-6-ylmethyl 2-hydroxy-3-methyl-2-phenylbutanoate hydrochloride (Compound No. 32),
-(1α, 5α, 6α)-3-azabicyclo[3.1.0]hex-6-ylmethyl 2-hydroxy-3-methyl-2-phenylbutanoate succinate (Compound No. 33),
-(1α, 5α, 6α)-3-azabicyclo[3,1.0]hex-6-ylmethyl 2-hydroxy-3-methyl-2-phenylbutanoate hydrobromide (Compound No. 34),
and their pharmaceutically acceptable solvates, esters, enantiomers, diastereomers, N-oxides, prodrugs, polymorphs and metabolites.

8. A pharmaceutical composition comprising a therapeutically effective amount of a compound of any one of the preceding claims together with pharmaceutically acceptable carriers, excipients or diluents.

9. A method for treatment or prophylaxis of an animal or a human suffering from a disease or disorder of the respiratory, urinary and gastrointestinal systems, wherein the disease or disorder is mediated through muscarinic receptors, comprising administering to said animal or human, a therapeutically effective amount of a compound of any one of the claims 1-7.

10. The method according to claim 9 wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

11. The method for treatment or prophylaxis of an animal or a human suffering from a disease or disorder of the respiratory, urinary and gastrointestinal systems, wherein the disease or disorder is mediated through muscarinic receptors, comprising administering to said animal or human, a therapeutically effective amount of the pharmaceutical composition according to claim 8.

12. The method according to claim 11 wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

13.-23. (canceled)
